(19) United States
(12) Patent Application Publication
(10) Pub. No.: US 2015/0265203 A1
(43) Pub. Date: Sep. 24, 2015

(54) Monitoring Antepartum Conditions Using a Smart Phone

(71) Applicants: Mark McConkie, Kaysville, UT (US); Cesare Jenkins, Stanford, CA (US)

(72) Inventors: Mark McConkie, Kaysville, UT (US); Cesare Jenkins, Stanford, CA (US)

(21) Appl. No.: 14/220,848

(22) Filed: Mar. 20, 2014

(51) Int. Cl. A61B 5/00

(52) U.S. Cl. CPC A61B 5/4356; A61B 5/6898; A61B 5/742; A61B 5/0022

(57) ABSTRACT

Antepartum conditions can be monitored using a smart phone or other portable user device. By providing a way to monitor antepartum conditions using a smart phone, the present invention allows virtually any user to monitor such conditions in any location without needing to visit a hospital or other health care facility. The ability to monitor antepartum conditions via a smart phone can therefore minimize false alarm trips to the hospital and can provide peace of mind to expectant mothers as they await active labor.

### CROSS-REFERENCE TO RELATED APPLICATIONS

Not Applicable.

### BACKGROUND

As childbirth approaches, it is common to monitor the occurrence of contractions in order to identify when active labor has begun. For example, many people use a clock to time the frequency of contractions that a woman is experiencing. Typically, once a certain frequency of contractions is identified, a woman, believing that she is in active labor, will go to the hospital to give birth. Oftentimes, once the woman is examined at the hospital, it is determined that the woman has incorrectly identified the occurrence of contractions and is not yet in active labor. In such cases, the woman is usually sent home until active labor has begun.

### BRIEF SUMMARY

The present invention extends to methods, systems, and computer program products for monitoring antepartum conditions using a smart phone or other portable user device. By providing a way to monitor antepartum conditions using a smart phone, the present invention allows virtually any user to monitor such conditions in any location without needing to visit a hospital or other health care facility. The present invention can therefore minimize false alarm trips to the hospital and can provide peace of mind to expectant mothers as they await active labor.

Monitoring antepartum conditions via a smart phone can be enabled by providing communications circuitry which interfaces a sensor with a smart phone. The communications circuitry can be incorporated into the sensor or into a separate component to which the sensor and smart phone connect whether via a wired or a wireless connection. The communications circuitry can be used to convert raw sensor data into an appropriate format for transmission to a smart phone. The communications circuitry can also be used to provide power to the sensor. In some cases, power can be provided by the smart phone to power the communications circuitry and/or the sensor.

An application executing on the smart phone can be employed to receive data from the communications circuitry and to display the data to the user. The displayed data can include representations of the current antepartum conditions being experienced as well as a summary of ongoing antepartum conditions. The application can also be configured to identify when the received data indicates that the expectant mother has entered active labor and can provide appropriate notifications. These notifications can include a prompt to the user to go the hospital, or a notification that is sent to a doctor or other health care professional.

In one embodiment, the present invention is implemented as a system for monitoring one or more antepartum conditions of an expectant mother. The system includes communications circuitry configured to receive a signal, from a sensor, that represents one or more antepartum conditions of an expectant mother, and to process the signal into a formatted signal that is transmittable to a smart phone. The system also includes an application, executable on a smart phone, that is configured to receive the formatted signal and display one or more representations of the one or more antepartum conditions on the smart phone.

In another embodiment, the present invention is implemented as a system for monitoring contractions experience by an expectant mother. The system includes communications circuitry configured to receive a signal, from a tocodynamometer transducer, that represents pressure sensed by the tocodynamometer transducer when attached to the abdomen of an expectant mother, the communications circuitry being further configured to process the signal into a formatted signal that is transmittable to a smart phone. The system also includes an application, executable on a smart phone, that is configured to receive the formatted signal and display, on the smart phone, one or more representations of the pressure sensed by the tocodynamometer transducer.

In another embodiment, the present invention is implemented as a method for monitoring one or more antepartum conditions of an expectant mother using a smart phone. A signal generated by a sensor is received by communications circuitry. The signal represents one or more antepartum conditions of an expectant mother. The communications circuitry processes the signal into a formatted signal that is transmittable to a smart phone. The formatted signal is then transmitted via a wired or wireless connection to the smart phone. The smart phone then displays one or more representations of the one or more antepartum conditions on the smart phone.

This summary is provided to introduce a selection of concepts in a simplified form that are further described below in the Detailed Description. This Summary is not intended to identify key features or essential features of the claimed subject matter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an example computing environment in which the present invention can be implemented;

FIG. 2 illustrates a first example embodiment in which communications circuitry is included within a sensor;

FIG. 3 illustrates an example of a prior art tocodynamometer transducer;

FIG. 4 illustrates a second example embodiment in which communications circuitry is included in a separate communications device;

FIG. 4A illustrates a variation of the second example embodiment in which the separate communications device attaches directly to a smart phone;

FIG. 5 illustrates an example user interface that can be displayed to the user;

FIG. 6 illustrates an example where the communications device of FIG. 4 can be attached directly to a smart phone via a port.

### DETAILED DESCRIPTION

The present invention extends to methods, systems, and computer program products for monitoring antepartum conditions using a smart phone or other portable user device. By providing a way to monitor antepartum conditions using a smart phone, the present invention allows virtually any user to monitor such conditions in any location without needing to visit a hospital or other health care facility. The present invention can therefore minimize false alarm trips to the hospital and can provide peace of mind to expectant mothers as they await active labor.

Monitoring antepartum conditions via a smart phone can be enabled by providing communications circuitry which interfaces a sensor with a smart phone. The communications circuitry can be incorporated into the sensor or into a separate component to which the sensor and smart phone connect whether via a wired or a wireless connection. The communications circuitry can be used to convert raw sensor data into an appropriate format for transmission to a smart phone. The communications circuitry can also be used to provide power to the sensor. In some cases, power can be provided by the smart phone to power the communications circuitry and/or the sensor.

An application executing on the smart phone can be employed to receive data from the communications circuitry and to display the data to the user. The displayed data can include representations of the current antepartum conditions being experienced as well as a summary of ongoing antepartum conditions. The application can also be configured to identify when the received data indicates that the expectant mother has entered active labor and can provide appropriate notifications. These notifications can include a prompt to the user to go the hospital, or a notification that is sent to a doctor or other health care professional.

In one embodiment, the present invention is implemented as a system for monitoring one or more antepartum conditions of an expectant mother. The system includes communications circuitry configured to receive a signal, from a sensor, that represents one or more antepartum conditions of an expectant mother, and to process the signal into a formatted signal that is transmittable to a smart phone. The system also includes an application, executable on a smart phone, that is configured to receive the formatted signal and display one or more representations of the one or more antepartum conditions on the smart phone.

In another embodiment, the present invention is implemented as a system for monitoring contractions experience by an expectant mother. The system includes communications circuitry configured to receive a signal, from a tocodynamometer transducer, that represents pressure sensed by the tocodynamometer transducer when attached to the abdomen of an expectant mother, the communications circuitry being further configured to process the signal into a formatted signal that is transmittable to a smart phone. The system also includes an application, executable on a smart phone, that is configured to receive the formatted signal and display, on the smart phone, one or more representations of the pressure sensed by the tocodynamometer transducer.

In another embodiment, the present invention is implemented as a method for monitoring one or more antepartum conditions of an expectant mother using a smart phone. A signal generated by a sensor is received by communications circuitry. The signal represents one or more antepartum conditions of an expectant mother. The communications circuitry processes the signal into a formatted signal that is transmittable to a smart phone. The formatted signal is then transmitted via a wired or wireless connection to the smart phone. The smart phone then displays one or more representations of the one or more antepartum conditions on the smart phone.

Embodiments of the present invention may comprise or utilize special purpose or general-purpose computers including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system.

Computer-readable media is categorized into two disjoint categories: computer storage media and transmission media. Computer storage media (devices) include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other similarly storage medium which can be used to store desired program code means in the form of computer-executable instructions or data structures and which can be accessed by a general purpose or special purpose computer. Transmission media include signals and carrier waves.

Computer-executable instructions comprise, for example, instructions and data which, when executed by a processor, cause a general purpose computer, special purpose computer, or special purpose processing device to perform a certain function or group of functions. The computer executable instructions may be, for example, binaries, intermediate format instructions such as assembly language or P-Code, or even source code.

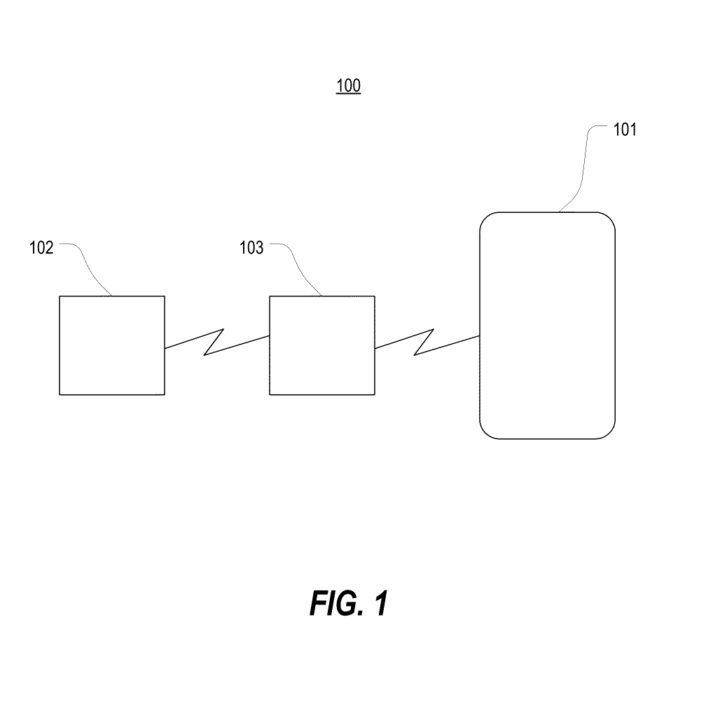

FIG. 1 illustrates an example computer environment 100 in which the present invention can be implemented. Computer environment 100 includes a smart phone 101, a sensor 102, and communications circuitry 103 that interfaces smart phone 101 with sensor 102. Sensor 102 comprises the necessary circuitry for creating a signal that represents one or more antepartum conditions of an expectant mother. Communications circuitry 103 can be configured to receive a signal output by sensor 102 and perform appropriate processing on the signal to allow the signal to be transmitted to smart phone 101. Smart phone 101 can therefore be configured to receive the processed signal from communications circuitry 103 and generate a display of information contained with the processed signal to allow a user to monitor the one or more antepartum conditions of the expectant mother via smart phone 101.

In this specification, a smart phone should be construed to include not only devices having cellular telephone capabilities, but should also include other similar portable user devices such as tablets or portable media players. The present invention can also be implemented with other computing devices such as laptops, desktop computers, and televisions. As will become apparent below, communications circuitry can be employed to enable many different types of general computing devices to receive and display sensor data representing one or more antepartum conditions of an expectant mother.

In some embodiments, smart phone 101 can include a mobile application that is configured to receive and process the signal from sensor 102 (via communications circuitry 103). Accordingly, a mobile application can be used to enable many different types of smart phones to act as systems for monitoring antepartum conditions. Due to the wide availability of such smart phones, virtually any user can monitor antepartum conditions from virtually any location without requiring the expectant mother to visit a hospital or other health care facility.

The present invention can be employed with various types of sensors for monitoring an antepartum condition. For example, sensor 102 can represent a tocodynamometer transducer, a fetal Doppler sensor, or an intrauterine pressure catheter. Other types of sensors can also be configured to communicate with smart phone 101 to allow smart phone 101 to monitor other antepartum conditions.

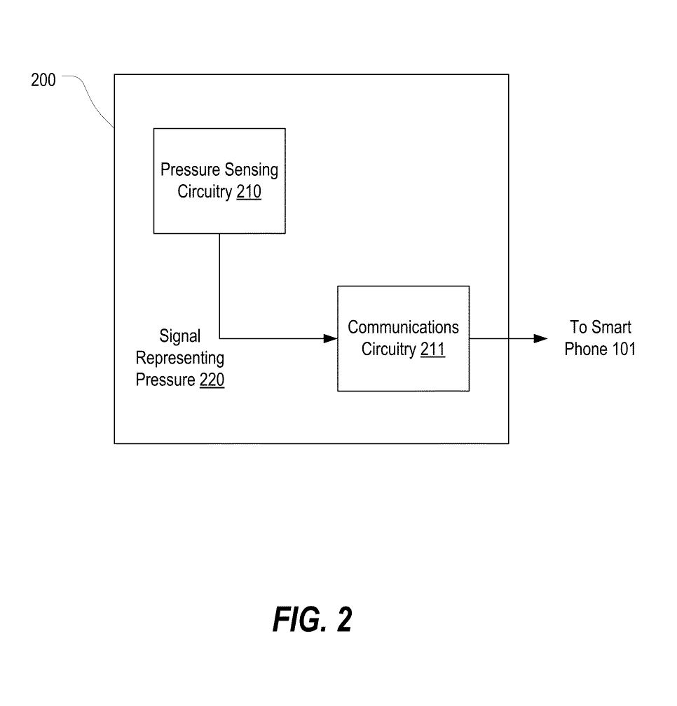

FIG. 2 illustrates a first example embodiment where the sensor is a tocodynamometer transducer 200. As shown, tocodynamometer transducer 200 includes pressure sensing circuitry 210 and communications circuitry 211. Pressure sensing circuitry 210 can comprise the necessary circuitry for representing variations in pressure sensed on the expectant mother's abdomen. For example, pressure sensing circuitry 210 can employ a resistance based or a capacitance based sensing element. In each case, the sensing element can be configured to output signal 220 which varies based on the movement of a component of the sensor caused by a contraction. Typically, signal 220 is in the form of an analog voltage; however, some sensors may be configured to output a variable current value. In any case, pressure sensing circuitry 210 outputs signal 220 that represents the current amount of force applied to tocodynamometer 200.

Signal 220 is then received by communications circuitry 211 which performs the necessary processing to allow signal 220, or values derived from signal 220, to be transmitted to smart phone 101. The type of processing performed by communications circuitry 211 will depend on the type of connection employed between tocodynamometer transducer 200 and smart phone 101 as will be further described below.

Accordingly, the present invention can be implemented using a tocodynamometer transducer 200 that includes both pressure sensing circuitry 210 and communications circuitry 211, and a standard smart phone 101. As an example, FIG. 2 can represent the case when a standard tocodynamometer transducer is modified to include communications circuitry 211 to allow the tocodynamometer transducer to transmit pressure data directly to smart phone 101.

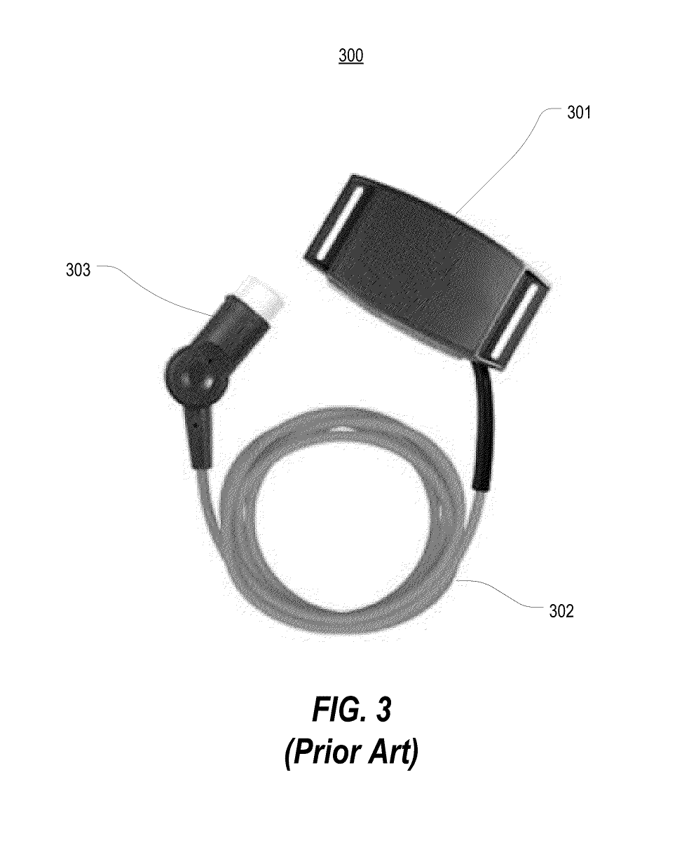

In this specification, a standard tocodynamometer transducer can be understood as the sensor that is typically placed on a woman's abdomen and that includes a cable through which the sensor both receives power and transmits signal 220. FIG. 3 provides an example of a standard tocodynamometer transducer 300. As shown, a standard tocodynamometer transducer includes a housing 301 which contains pressure sensing circuitry, a cable 302 over which power is supplied to the pressure sensing circuitry and data is transmitted to a dedicated monitoring system, and a connector 303 for connecting the standard tocodynamometer transducer to the dedicated monitoring system.

In contrast to a standard tocodynamometer, embodiments of the present invention as shown in FIG. 2 can provide a tocodynamometer transducer that includes communications circuitry 211 to allow the transducer to communicate directly with a smart phone. In some embodiments, communications circuitry 211 can include circuitry for enabling the transmission of signal 220 (or data derived from signal 220) wirelessly to smart phone 101 (e.g. via Bluetooth, Wi-Fi, NFC, etc.). In other embodiments, communications circuitry 211 can include circuitry for enabling the transmission of signal 220 over a wired connection to smart phone 101 (e.g. via an audio port, USB port, Apple's 30-pin port or Lighting port, PDMI port, etc.). In either case, communications circuitry 211 can perform appropriate processing on signal 220 to allow the signal (or data derived from the signal) to be transferred to smart phone 101 where it can ultimately be displayed within a mobile application.

In embodiments where either a wired or wireless connection is employed, a separate power source may be provided to power the circuitry of tocodynamometer transducer 200. For example, tocodynamometer transducer 200 may include a battery or a power cord for connecting to an electrical outlet. However, in some embodiments that employ a wired connection, tocodynamometer transducer 200 can receive power from smart phone 101 over the wired connection.

Accordingly, unlike a standard tocodynamometer transducer, tocodynamometer transducer 200 can be used without any dedicated monitoring system by simply connecting the transducer (whether by wire or wirelessly) to smart phone 101. Communications circuitry 211 can process signal 220 into an appropriate format that smart phone 101 can receive and ultimately display within a mobile application.

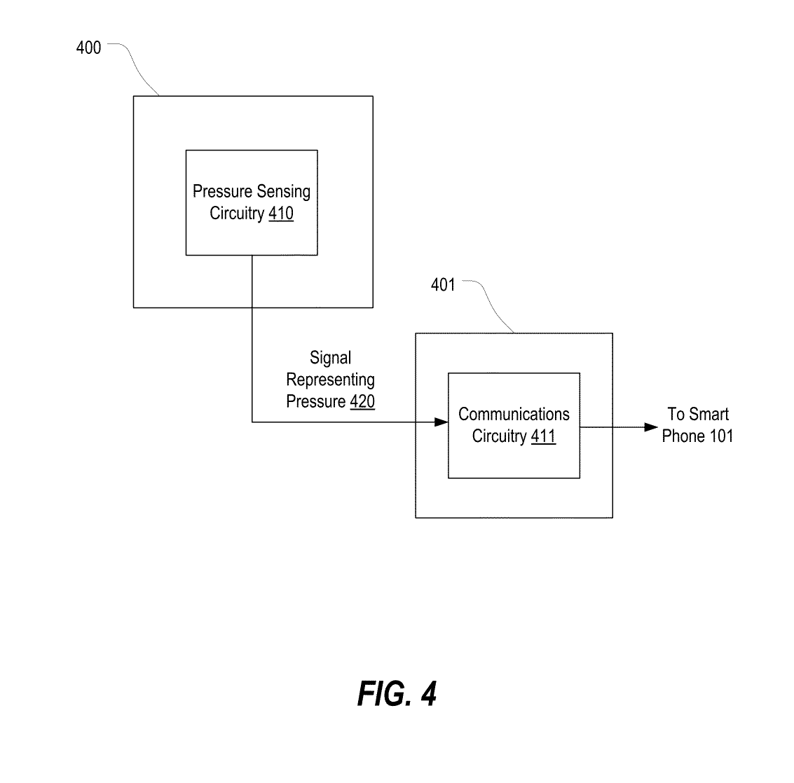

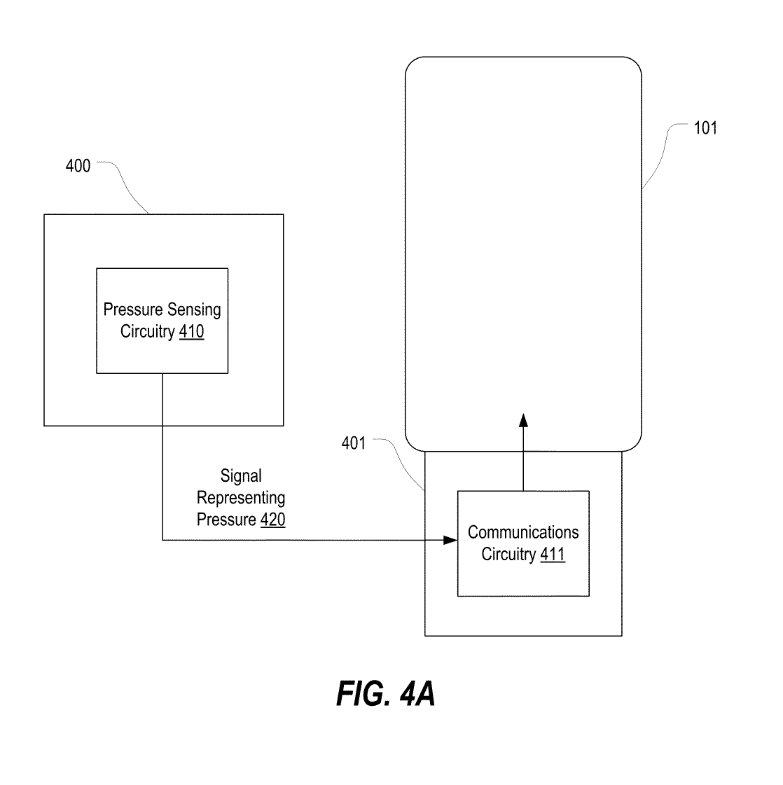

FIG. 4 illustrates a second example embodiment where a separate communications device 401 is employed for processing and transmitting a signal 420 received from a tocodynamometer transducer 400. This second embodiment may be preferred when tocodynamometer transducer 400 is a standard tocodynamometer transducer such as transducer 300. In such cases, communications device 401 can be configured to receive signal 420, process it into an appropriate format, and transmit it to smart phone 101.

For example, communications device 401 can include one or more ports to which tocodynamometer transducer 400 connects for providing signal 420 to communications device 401. Communications circuitry 411 can be similar to communications circuitry 211. In other words, communications circuitry 411 can receive signal 420 and perform appropriate processing to allow signal 420 (or data derived from signal 420) to be transmitted via a wired or wireless connection to smart phone 101.

Communications device 401 can include a power source (e.g. a battery or a power cord) for powering communications circuitry 411. In some embodiments, communications device 401 can also provide power to tocodynamometer transducer 400. Further, in some embodiments where communications device 401 employs a wired connection with smart phone 101, communications device 401 may not include a separate power source, but may be configured to receive power from smart phone 101. In such cases, the power from smart phone 101 can also be used to power tocodynamometer transducer 400.

In some embodiments, communications device 401 can be configured to transmit signal 420 directly to a server rather than to smart phone 101. In such cases, the server can receive signal 420 and process it in a suitable manner to allow the pressure data contained within signal 420 to be made accessible to smart phone 101. In other words, a server can be used as an intermediary between communications device 401 and smart phone 101. In such cases, smart phone 101 can include a mobile application that obtains pressure data from the server and then displays it on smart phone 101 to the user. Such embodiments can also facilitate sharing the pressure data with more than one user (e.g. by making the pressure data available to any mobile application having appropriate credentials).

FIG. 4A illustrates a variation of the second example embodiment in which communications device 401 attaches directly to smart phone 101. Therefore, in contrast to the embodiments described with respect to FIG. 4 that employ a wired connection, no intermediate wires are required between communications device 401 and smart phone 101. Communications device 401 can be configured with an appropriate adapter to allow communications device 401 to be plugged into a port of smart phone 101. For example, communications device 401 can include an audio plug for connecting to an audio jack, a USB connector for connecting to a USB port, an Apple Lighting connector for connecting to a Lighting port, or another similar connector that is commonly employed for connecting to smart phones or other mobile devices.

In any of the above described embodiments, once smart phone 101 receives pressure data (i.e. signal 220/420 or data derived therefrom), smart phone 101 can process, store, display, or further transmit the pressure data. For example, a mobile application executing on smart phone 101 can be configured to plot the pressure data for display to the user. Further, the mobile application can provide additional information about contractions that a woman may be experiencing such as the duration, frequency, and relative strength of the contractions. In this way, the user can monitor contractions using smart phone 101.

In some embodiments, the mobile application can be configured to transmit some or all of the pressure data to another computing system. For example, the mobile application can be configured to transmit the pressure data to a doctor or other health care professional. In some cases, the mobile application can be configured to identify from the pressure data when a woman has commenced active labor (e.g. by detecting a duration or frequency of contractions in excess of some threshold) and notify the doctor accordingly. Such notifications can be transmitted using any of the available protocols for communicating over a network such as the internet. For example, the mobile application can be configured to send an email or text message to the doctor, or to send a communication directly to a dedicated monitoring system at a health care facility.

In some embodiments, the pressure data received by smart phone 101 can be in the form of raw sensor data. For example, the pressure data can be an analog voltage signal (e.g. a minimally processed version of signal 220/420). In such cases, the mobile application can be configured to process the raw sensor data into a usable format such as by converting the raw sensor data into digital values (e.g. using the underlying hardware and/or operating system of the smart phone) and assigning timestamps to each value. In other embodiments, the pressure data can already be in a digital format with corresponding time stamps (such as if communications circuitry 211/411 includes an analog-to-digital converter).

Whether the pressure data received by smart phone 101 is raw sensor data or previously processed data, the mobile application can be configured to process the pressure data to identify changes in the pressure data that are indicative of the beginning and end of a contraction. For example, if a series of pressure data readings indicate a sharp increase or decrease in pressure, the mobile application can identify the beginning or end of a contraction respectively and notify the user accordingly. For example, each time the beginning of a contraction is identified, the mobile application can output an appropriate indication such as by updating the display of a user interface, outputting a sound, etc.

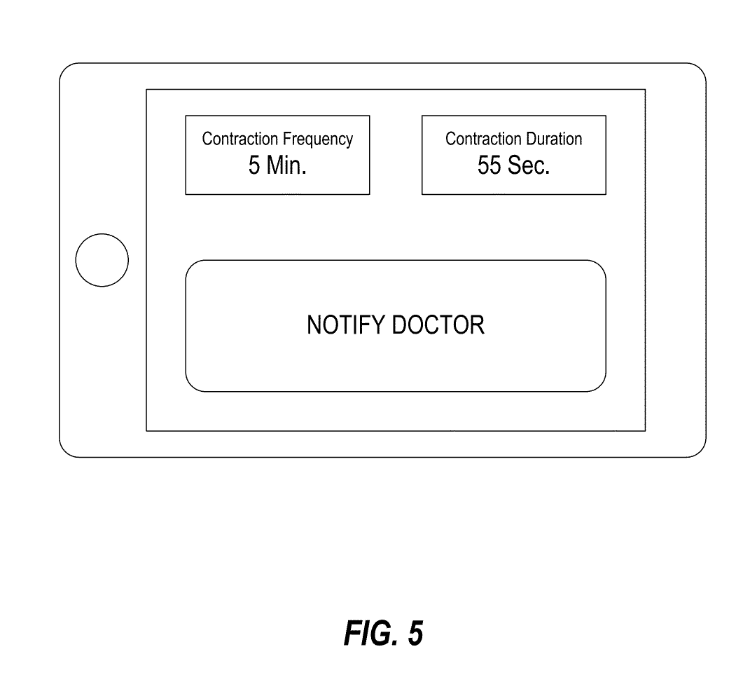

The mobile application can maintain a history of contraction start and stop times to allow it to provide appropriate summary information to the user or a health care professional. FIG. 5 provides an example user interface that the mobile application can display to the user. As shown, the user interface provides a current contractions frequency and a current contraction duration. These values can be generated based on the history of pressure data maintained by the mobile application. The user interface can also provide a button that can be selected to automatically contact a doctor or health care professional. In some embodiments, this button can be displayed only once the contraction frequency and duration indicate that the woman is likely in active labor. In other embodiments, the mobile application can provide a prompt to the user to recommend contacting the doctor or going to the hospital once the contraction frequency and duration indicate that active labor has begun.

Accordingly, the mobile application can assist the user in knowing when it is appropriate to visit the hospital and/or contact a doctor. Specifically, because the mobile application can provide a more accurate indication of the duration and frequency of contractions and because the mobile application can provide more reliable recommendations for when to visit the hospital, the user will likely make fewer false alarm trips to the hospital than when using traditional manual techniques for identifying active labor. The present invention can therefore minimize the costs associated with giving birth.

In some embodiments, the mobile application (or server when a server is used as an intermediary) can be configured to transmit the pressure data, or data derived from the pressure data, to a doctor or health care facility. As described above, this pressure data can be transmitted in response to user input or can be transmitted automatically, whether periodically or continuously. In this manner, the doctor or health care facility can monitor the status of the expectant mother and contact the expectant mother when desired or necessary. In some embodiments, the mobile application can be configured to receive and display such communications from the doctor or health care facility.

For example, by enabling the mobile application to transmit pressure data to a remote individual (whether a doctor, nurse, or other user), and receive communications back from the remote individual, the present invention can facilitate remote labor coaching. As the remote individual receives and reviews the expectant mother's pressure data in real time, the remote individual can send feedback, encouragement, or tips that can be displayed by the mobile application to assist the expectant mother during labor.

As another example, the mobile application (or a server when a server is used as an intermediary) can be configured to upload pressure data to a social networking site to allow many different individuals to view the expectant mother's labor status. Such uploads can occur continuously or can be made only when the contractions reach a particular milestone (e.g. every ten minutes, five minutes, three minutes, etc.). The mobile application can also be configured to receive and display comments provided by members of the social networking site after viewing the labor status.

Although the above discussion has focused primarily on tocodynamometer transducers, the present invention can also be implemented in conjunction with other types of sensors. For example, the same techniques can be employed to interface a fetal Doppler sensor or an intrauterine catheter with communications circuitry (e.g. 211/411) to allow the sensor data produced by such sensors to be received and processed by a smart phone.

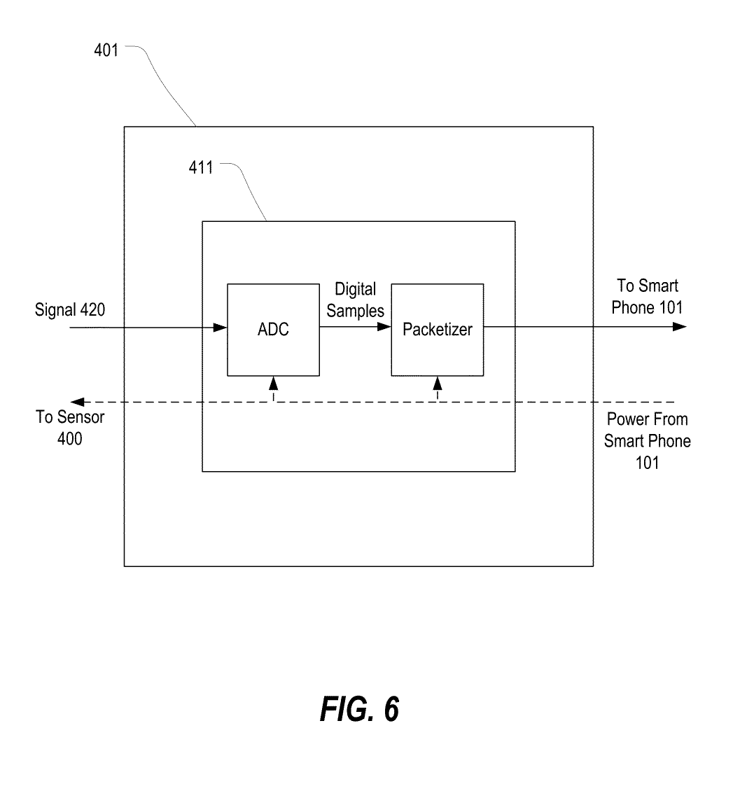

FIG. 6 provides an example implementation of communications device 401 when communications device is configured to connect to smart phone 101 via a wired connection. In this implementation, communications device 401 can connect to smart phone 101 via an intermediate cable (e.g. similar to FIG. 4) or via a direct connection to a port of smart phone 101 (e.g. similar to FIG. 4A).

As shown in FIG. 6, signal 420 received from sensor 400 can comprise a variable analog value. In such cases, communications circuitry 411 can include an analog-to-digital converter (ADC) that receives the analog value and outputs digital samples. These digital samples can be provided to a packetizer that formats the digital samples according to the type of connection employed between communications device 401 and smart phone 101. For example, if Bluetooth is employed, packetizer can comprise the components for creating and transmitting Bluetooth packets containing the digital samples.

As also shown in FIG. 6, in some embodiments, power can be provided from smart phone 101 to power the components of communications circuitry 411 and possibly sensor 400. The power is shown in a dashed line to represent that it may or may not be provided to communications circuitry 411 and/or sensor 400 depending on the specific implementation employed.

Although not shown, additional components can be provided within communications circuitry to, for example, assist in cleaning up the analog signal, synchronize the execution of the various components, buffer digital samples, etc. In embodiments where signal 420 is digital, an ADC would not be required and signal 420 could be provided to the packetizer for appropriate formatting.

In another embodiment, smart phone 101 can serve as a tocodynamometer transducer by using a screen that can detect variations in pressure. In such embodiments, smart phone 101 can be secured (e.g using an elastic strap) directly on the expectant mother's abdomen with the screen facing down. When a contraction occurs, the smart phone can sense the changes in pressure on the screen and generate pressure data accordingly. The pressure data can then be processed, transmitted, and/or displayed as described above. Accordingly, in such embodiments, a tocodynamometer can be implemented using only a smart phone with a pressure sensitive screen.

In some implementations, an additional component can be used that limits the area of the screen to which pressure is applied. In some cases, limiting the area of the screen that receives pressure can increase the accuracy of the reading. For example, a ring-shaped, circle-shaped, or square-shaped component could be positioned between the screen and the woman's abdomen to limit contact to the area of the component. Any displacement of the component caused during a contraction could be sensed by the pressure sensitive screen and converted into pressure data representing the occurrence of a contraction.

In another embodiment, smart phone 101 can serve as a tocodynamometer transducer even when its screen is not a pressure sensitive touch screen. In such embodiments, the touch screen (e.g. a capacitive touch screen) can be used to sense variations in the area of the screen that is in contact with the woman's abdomen. For example, prior to a contraction, the woman's abdomen where the phone is positioned may be relatively flat. As a result, a large percentage of the screen's surface area will be in contact with the woman's abdomen. Then, when a contraction occurs, the woman's abdomen may become more round resulting in a smaller percentage of the screen's surface area being in contact with the woman's abdomen. The capacitive touch screen can be used to detect these variations in the amount of the screen that is in contact with the woman's abdomen. Appropriate processing can be performed to translate these variations into pressure data representing the occurrence of a contraction. In some implementations, one or more straps or other supporting means for retaining the phone in a fixed position on the woman's abdomen can be used to prevent false reading caused by phone movement. In some implementations, the smart phone may contain one or more accelerometers or gyroscopes which can be used to sense significant movement of the phone (e.g. when the phone is repositioned on the woman's abdomen, or when the woman may be moving substantially while the phone is on her abdomen) and account for such movement to prevent the movement from generating false indications of contractions.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description.

What is claimed:

1. A system for monitoring one or more antepartum conditions of an expectant mother, the system comprising:
communications circuitry configured to receive a signal, from a sensor, that represents one or more antepartum conditions of an expectant mother, and to process the signal into a formatted signal that is transmittable to a smart phone; and
an application, executable on a smart phone, that is configured to receive the formatted signal and display one or more representations of the one or more antepartum conditions on the smart phone.

2. The system of claim 1, further comprising:
a sensor that contains the communications circuitry.

3. The system of claim 1, further comprising:
a communications device that contains the circuitry.

4. The system of claim 3, wherein the communications device is configured to attach directly to a port of the smart phone.

5. The system of claim 4, wherein the port is one of: an audio port, or a port that provides digital input to the smart phone.

6. The system of claim 1, wherein the communications circuitry is connected to the smart phone via a wired or a wireless connection.

7. The system of claim 1, wherein the communications circuitry is connected to the smart phone via a wired connection, the communications circuitry receiving power over the wired connection.

8. The system of claim 7, wherein the communications circuitry is configured to provide power to the sensor.

9. The system of claim 1, wherein the signal is an analog signal.

10. The system of claim 9, wherein the formatted signal comprises digital values generated from the analog signal, the digital values having timestamps.

11. The system of claim 1, wherein the signal comprises pressure data received from a tocodynamometer transducer, and wherein the one or more representations displayed on the smart phone comprise a representation of one or more of a frequency or a duration of contractions.

12. The system of claim 1, wherein the application is further configured to transmit at least a portion of the formatted signal to a computing system at a health care facility.

13. The system of claim 12, wherein the at least a portion of the formatted signal is transmitted in response to either:
determining that the one or more antepartum conditions indicate that active labor has commenced; or
receiving user input that requests that the at least a portion of the formatted signal be transmitted.

14. The system of claim 1, wherein the signal comprises data received from a fetal Doppler sensor or from an intrauterine pressure catheter.

15. A system for monitoring contractions experienced by an expectant mother, the system comprising:
communications circuitry configured to receive a signal, from a tocodynamometer transducer, that represents pressure sensed by the tocodynamometer transducer when attached to the abdomen of an expectant mother, the communications circuitry being further configured to process the signal into a formatted signal that is transmittable to a smart phone; and
an application, executable on a smart phone, that is configured to receive the formatted signal and display, on the smart phone, one or more representations of the pressure sensed by the tocodynamometer transducer.

16. The system of claim 15, wherein the communications circuitry is contained within a communications device that connects to the smart phone via a wired or wireless connection.

17. The system of claim 16, wherein the wired connection comprises a direct connection of the communications device to a port of the smart phone.

18. The system of claim 15, wherein the communications circuitry is configured to provide power to the tocodynamometer transducer.

19. A tocodynamometer comprising:
a smart phone having a touch screen, the touch screen configured to detect variations in pressure when the touch screen is placed on a woman's abdomen, and to output a representation of the variations.

20. The tocodynamometer of claim 19, wherein the touch screen is one of a pressure sensitive touch screen or a capacitive touch screen.
